(19) United States
(12) Patent Application Publication
(10) Pub. No.: US 2020/0297522 A1
(43) Pub. Date: Sep. 24, 2020

(54) PATIENT-SPECIFIC PLANTAR PRESSURE RELIEF

(71) Applicant: Charles MILGROM, Moshav Aminadav (IL)

(72) Inventors: Charles MILGROM, Aminadav (IL); Gregory KLEIN, Kiriat Motzkin (IL)

(21) Appl. No.: 16/766,036

(22) PCT Filed: Nov. 21, 2018
(86) PCT No.: PCT/IL2018/051264

§ 371 Date: May 21, 2020

Related U.S. Application Data
(60) Provisional application No. 62/588,974, filed on Nov. 21, 2017.

(51) Int. Cl. A61F 5/14; A61F 5/30; A61B 5/103; A61B 5/107

(52) U.S. Cl. CPC A61F 5/14; A61B 5/1077; A61B 5/1038; A61F 5/30

(57) ABSTRACT

A patient specific device for reducing plantar pressure, a system and a method for manufacturing the same are disclosed. The method includes: obtaining pressure measurements of a plurality of plantar regions of a patient, wherein said measurements are acquired from at least one hardware pressure sensor; providing patient specific parameters for manufacturing a patient specific device, wherein the patient specific parameters comprise ranges of durometers for materials suitable for engaging each of the plurality of plantar regions based on a plurality of rules and manufacturing the patient specific device. Also disclosed is an insole adaptive layer (IAL), including a surface including a plurality of contour lines, the contour lines defining zones, wherein the surface within each zone having predetermined mechanical properties specific to the zone and the contour lines of each of the surface zones correspond to contour lines obtained from pedobarographic data of a subject.

### TECHNICAL FIELD

The present invention relates to the field of orthotics and more particularly, but not exclusively, to orthotic insoles of shoes.

### BACKGROUND

Plantar pressure can lead directly to undesirable injury and symptoms in the foot. Such injury or symptoms may include pain in a foot with sensation, or tissue damage and ulceration in a foot without sensation. As a result, reducing pressure at identified high pressure locations is believed to offer a therapeutic strategy for treatment of foot disorders.

Plantar ulcers are a source of concern for many diabetic patients. The ulcers are often caused by continuous elevated pressure that may be the result of, e.g., abnormal gait pattern, foot deformity, a foreign object, etc. The problem is made more acute by the loss of protective sensation and may be exacerbated by reduced blood flow found in the extremities of some diabetic patients.

Attempts to address the problem of plantar ulcers focus on off-loading the affected areas such that the pressure on the ulcerated area is reduced or eliminated during ambulatory activity. One of the means to prevent diabetic foot ulcers is the use of shoe orthotics or inserts which are made to relieve the pressure in areas of excessive pressure on the sole of the foot.

An exact pressure map of the foot while standing can be obtained using plantar pressure measuring systems. In some systems the measurements are made standing or walking while barefoot. Other systems can also measure plantar pressures within shoes while ambulating using in-shoe sensors.

Systems that map plantar pressure usually are composed of sensor elements. The actual sensors can be either capacitive, resistive, piezoelectric or piezoresistive. Some systems report the actual numerical pressure at each sensor point, either in kilo Pascals or kilo Pascals/cm² or expressed within specific pressure ranges. The pressure ranges are often expressed in a color scale. In some systems the pressure values are normalized and expressed as a percentage of the maximum pressure and maybe presented divided into switching levels. In some systems the pressure values presented are not for individual sensors but rather in predetermined larger areas of the foot. An example of a color representation of a standing plantar pressure foot map is shown in FIG. 1.

Some plantar foot pressure measuring systems can make a dynamic plantar pressure map while walking. These system present pressures either present data as maximum pressure maps, cumulative pressure maps or average pressure maps. Often the plantar pressure map is presented in colors, with corresponding pressure values of the colors presented in color alongside the map.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with an embodiment, a method for manufacturing a patient specific device for reducing plantar pressure in a patient, the method comprising the steps of: obtaining pressure measurements of a plurality of plantar regions of a patient, wherein said measurements are acquired from at least one hardware pressure sensor; providing patient specific parameters for manufacturing a patient specific device, wherein the patient specific parameters comprise durometer ranges for materials suitable for each of the plurality of plantar regions based on a plurality of rules; and manufacturing the patient specific device.

Optionally, the plurality of regions is selected from the group consisting of: medial heel, lateral heel, medial midfoot, lateral midfoot, first metatarsal, second metatarsal, lateral metatarsal, hallux, second toe, lateral toe.

Optionally, the manufacturing is by a 3D printer.

Optionally, the method is for manufacturing shoe insole. Alternatively, the method is for manufacturing forefoot extension.

Optionally, the patient specific device comprises at least one region configured to contact one or more plantar regions selected from the group consisting of: medial heel, lateral heel, medial midfoot, lateral midfoot, first metatarsal, second metatarsal, lateral metatarsal, hallux, second toe, lateral toe.

There is provided, in accordance with an embodiment, an insole adaptive layer (IAL), including a surface including a plurality of contour lines, the contour lines defining zones, wherein the surface within each zone having predetermined mechanical properties specific to the zone and the contour lines of each of the surface zones correspond to contour lines obtained from pedobarographic data of a subject. In some embodiments, the mechanical properties include one or more or a combination of Shore hardness, young's modulus, shear modulus, yield stress, compression, thickness, elasticity, and ductility. In some embodiments, one or more of the surface zones is configured to succumb to pressure applied by a corresponding plantar pressure zone of a subject.

According to some embodiments, values of the mechanical properties of one or more zone of the surface are inversely related to the pressure values of one or more region in the pedobarographic data. In some embodiments, one or more of the surface zones succumbs to pressure applied by a corresponding plantar pressure zone of a subject by deformation of the insole adaptive layer surface within the zone. In some embodiments, one or more of the zones is least deformable and supports over 50% of the plantar pressure applied by a sole of a subject. In some embodiments, a degree of deformation of the surface within the surface zone corresponds to a value of pressure applied by the plantar pressure zone as expressed by the pedobarographic data of the subject.

In some embodiments, the mechanical properties of each zone vary throughout a depth of the zone. In some embodiments, one or more of the zones is sized such that a corresponding plantar zone of a subject placed on the insole adaptive layer remains engaged with the insole adaptive layer zone during shifting of the plantar zone in respect to the insole adaptive layer. In some embodiments, the pedobarographic data includes a graphic plantar map. In some embodiments, the surface zones vary in thickness. In some embodiments, the surface is composed of one or more materials.

In some embodiments, the insole adaptive layer includes a top layer over the surface. In some embodiments, one or more materials has a Shore hardness in the range of 00-10 to A-60. In some embodiments, one or more materials has a Shore hardness in the range of A-50-10 to D-100. In some embodiments, the surface includes one or more layered materials, having a cumulative Shore hardness of one or more of A-27, A-40, A-50, A-60, D-0, D-10, A-70, D-20, A-85, A-9. In some embodiments, the surface includes one or more materials arranged in layers of varying thicknesses, having a cumulative Shore hardness of one or more of A-27, A-40, A-50, A-60, D-0, D-10, A-70, D-20, A-85, A-9.

There is provided, in accordance with an embodiment, a method for manufacturing an insole adaptive layer, including generating pedobarographic data of a sole of a subject applied to a surface, recording zones defined by the pedobarographic data, designating Shore hardness values to the zones in accordance with the pedobarographic data, generating a model associating the designated Shore hardness values with the recorded zones and printing a surface of an insole adaptive layer including zones of different Shore hardness in accordance with the model. In some embodiments, printing one or more layer using a plurality of materials.

There is provided, in accordance with an embodiment, a system for manufacturing an insole adaptive layer including one or more sensor used to obtain pedobarographic data of a subject, a processor in communication with the sensor, a manufacturing device operative to manufacture the insole adaptive layer. In some embodiments, the processor includes a computer program product configured to record zones defined by the pedobarographic data and designate Shore hardness values to the zones in accordance with the pedobarographic data and generate a model associating the designated Shore hardness values with the recorded zones and wherein the manufacturing device is configured to produce a surface of an insole adaptive layer including zones of different Shore hardness in accordance with the model.

According to some embodiments, the manufacturing device is a 3D printer. In some embodiments, the manufacturing device is configured to produce zones in which mechanical properties of the surface within the zone are distinct.

There is provided, in accordance with some embodiments, an insole adaptive layer, including a surface including a plurality of zones, wherein the mechanical properties of the surface being specific to each of the zones and the surface within the zones is configured to succumb to pressure applied by a corresponding plantar pressure zone on a subject's sole when placed on the insole adaptive layer.

There is provided, in accordance with some embodiments, plantar data for manufacturing an IAL is obtained using one or more of pedobarography and non-pressure related scans. In some embodiments, the non-pressure related scans include one or more of thermal imaging, ultrasound, x-ray, CT-scans, Mill, and spectroscopy. In some embodiments, the plantar data obtained by the non-pressure related scans is converted to pedobarographic data. In some embodiments, the data obtained from the non-pressure related scans is converted by a computer algorithm to mechanical properties values or data that indicate contour lines of the surface zones of the IAL. In some embodiments, the converted data is communicated to a manufacturing device.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a flow chart of a method for designing and manufacturing an improved patient specific pressure reducing device;

FIG. 2 is a block diagram of a system for manufacturing a patient specific device for reducing plantar pressure, in accordance with an embodiment;

FIG. 3 is a schematic illustration of a patient specific device for reducing plantar pressure, in accordance with an embodiment;

FIG. 4 is a top view simplified illustration of one embodiment of an insole adaptive layer;

FIGS. 5A-B are top view simplified illustrations of one embodiment of an insole adaptive layer corresponding with data of a pedobarographic measurement;

FIG. 6 is a system for producing an insole adaptive layer;

FIG. 7 is a flow chart for a method for manufacturing an insole adaptive layer;

FIG. 8 is a side view simplified illustration of one embodiment of a shoe insole comprising an insole adaptive layer;

FIGS. 9A-C are top view simplified illustrations of some embodiments of an insole adaptive layer; and

FIGS. 10A-C are cross section views of simplified illustrations of some embodiments of an insole adaptive layer.

### DETAILED DESCRIPTION

A patient specific device for reducing plantar pressure in a patient in need thereof is disclosed herein. Also disclosed is a method for planning and manufacturing the device. The patient specific device is an Insole Adaptive Layer (IAL) designed to be applied onto either a full insole—one which extends to the full size of a patient's foot sole, or a partial insole—one which extends only to a portion of the size of the patient's foot sole, for example a forefoot extension. The insole being fixed inside a shoe or removable therefrom.

The patient specific Insole Adaptive Layer (IAL) is designed according to patient specific parameters. These parameters may be provided by computer software that analyzes regional peak pressure. For a non-limiting example, patient specific parameters may be generated using one or more pressure sensors. In another example, a system that maps plantar pressure is used. Typically, systems that map plantar pressure are composed of sensor elements. The actual sensors can be either capacitive, resistive, piezoelectric or piezoresistive, to name a few examples. Some systems report the actual numerical pressure at each sensor point, either in kilo Pascal or kilo Pascal/cm², or expressed within specific pressure ranges. Commercially available systems include, for example: the F-Scan System by Tekscan, Inc, of Boston, Mass.; and the Stepscan® Pedway by ViTRAK Systems Inc., Canada. In some embodiments, the pedobarographic data is obtained from a 3D scanner, e.g., Rscan® by RSscan International NV, Belgium.

The terms “patient” or “subject” are used interchangeably herein and refer to a human subject suffering from plantar pain and/or plantar ulcers. The term also encompasses human subjects which are at risk of suffering from plantar ulcers such as subjects suffering from diabetes and/or subjects suffering from neuropathy. Further yet, the term encompasses human subjects which are otherwise in need of the patient-specific IAL of present embodiments.

For purposes of clarity of explanation, the IAL described throughout this disclosure relates to as a device used to provide relief to diabetic patients by unloading pressure off plantar areas that may include diabetic sores (e.g., patients suffering from plantar ulcers). However the IAL described herein can be used for a multitude of medical conditions in which unloading plantar pressure from a subject is desired. For example, subjects suffering from neuropathy, plantar corns, calluses, warts, plantar fasciitis, interdigital neuroma, fistulous tracts, lesions, or pain caused by foreign bodies or abnormalities.

Throughout this disclosure manufacturing of the IAL is based on pedobarographic data, however, data for manufacturing an IAL can be obtained via non-pressure related scanning methods, such as, for example, thermal imaging of plantar surface of the subject, ultrasound, x-ray, CT-scans, Mill and spectroscopy. Such scans can be used to identify areas from which unloading pressure is indicated, for example, information regarding deep subcutaneous plantar areas which can appear normal on visual observation or when pedobarographic data is obtained using standard pressure sensors. The information obtained from such non-pressure scans can be converted by a computer algorithm to pedobarographic data, or alternatively, can be converted to mechanical properties values or data indicating contour lines of the surface zones of the IAL and communicated to a manufacturing device (e.g., device processor).

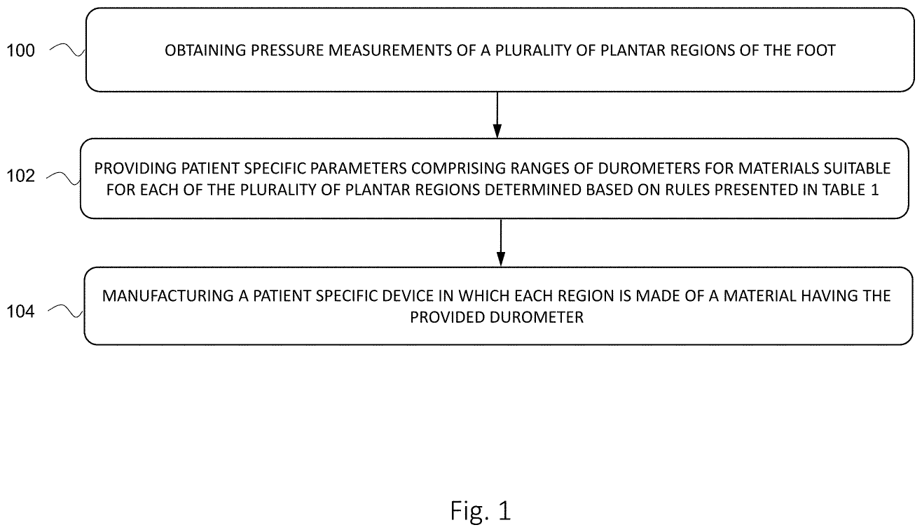

Reference is now made to FIG. 1 which is a flow chart of a method for manufacturing a patient specific device for reducing plantar pressure in a patient. Pressure measurements of a plurality of plantar regions of the foot are obtained (step 100). Optionally, other measurements are obtained to generate a computer model of the foot. Optionally, one or more pressure sensors may be used to compute the pressures of a plurality of plantar regions. Optionally, the plantar region of the foot may be scanned using digital photos, videos, laser scanning, etc. Optionally, the pressure measurements include an analysis of pressure distribution across the plantar foot during engagement in certain activities, such as walking or running. Optionally, a computer program that provides a plantar pressure map may be utilized. Optionally, the plurality of regions are selected from the group consisting of: medial heel, lateral heel, medial midfoot, lateral midfoot, first metatarsal, second metatarsal, lateral metatarsal, hallux, second toe, and lateral toe. Further optionally, a region of the plurality of regions may encompass two or more of the group members listed above.

Patient specific parameters for manufacturing a patient specific device are computed (step 102). Optionally, the patient specific parameters include durometer values or durometer ranges of materials suitable for each of a plurality of regions of the device, which are corresponding to the plurality of plantar regions. As used herein, a “region” of a device which is corresponding to a plantar region, refers to a specific region of the device configured to contact and/or support a specific plantar region. Optionally, a durometer map is provided.

For this purpose, a computer program that computes a suitable durometer range for a material for each of the plurality of plantar regions based on a plurality of rules, may be used. Optionally, the suitable durometer range for each region may be computed directly from the pressure measurements of the plurality of plantar region of the foot, or may further include physician input (e.g., as to a region that has been the subject of a previous lesion or of a current lesion). Optionally, pressure measurements may be compared to threshold pressure values for each plantar region. Optionally, a degree of load pressure for a plantar region is determined based on a deviation of the measured pressure from threshold pressure value for each plantar region. In an exemplary embodiment, a degree of load pressure of each of the plurality of plantar regions is determined according to the relationships shown on Table 1. Optionally, a durometer range for a material suitable for a device region is computed based on a deviation of the pressure of a corresponding plantar region from the threshold value for the corresponding plantar region. As a non-limiting example, thresholds values may be predefined by measuring and analyzing plantar pressure of normal subjects and sick subjects (e.g., subjects suffering from plantar pain and/or plantar ulcers). As a non-limiting example, the percent of deviation of a pressure from a threshold pressure in a specific region is used to compute the durometer of the material suitable for a region of the device corresponding to the plantar region.

TABLE 1
Degree of load pressure for each plantar region

| | | Extra | | | | Extra |
|---|---|---|---|---|---|---|
| | Load pressure | low | Low | Medium | High | high |
| Medial | Load in Kilo Pascal | <76.1 | 76.1-107.5 | 107.5-170.3 | 170.3-201.7 | >201.7 |
| heel | % of total foot load | <15.3 | 15.3-23.9 | 23.9-41.1 | 41.1-49.7 | >49.7 |
| Lateral | Load in Kilo Pascal | <68 | 68-100.3 | 100.3-164.9 | 164.9-197.2 | >197.2 |
| heel | % of total foot load | 11.6 | 11.6-19.8 | 19.8-36.2 | 36.2-44.4 | >44.4 |
| Medial | Load in Kilo Pascal | — | 0-5.7 | 5.7-32.7 | 32.7-46.2 | >46.2 |
| midfoot | % of total foot load | — | 0-0.3 | 0.3-2.5 | 2.5-3.6 | >3.6 |
| Lateral | Load in Kilo Pascal | — | 0-13.2 | 13.2-42.4 | 42.4-57 | >57 |
| midfoot | % of total foot load | — | 0-1.5 | 1.5-11.3 | 11.3-16.2 | >16.2 |
| First | Load in Kilo Pascal | — | 0-14.4 | 14.4-62.4 | 62.4-86 | >86.4 |
| metatarsal | % of total foot load | — | 0-1.1 | 1.1-10.1 | 10.1-14.6 | >14.6 |
| Second | Load in Kilo Pascal | <8.6 | 8.6-30.2 | 30.2-73.4 | 73.4-95 | >95 |
| metatarsal | % of total foot load | <0.8 | 0.8-4.6 | 4.6-12.2 | 12.2-16 | >16 |
| Lateral | Load in Kilo Pascal | <9.8 | 9.8-31.6 | 31.6-75.2 | 75.2-97 | >97 |
| metatarsal | % of total foot load | <0.9 | 0.9-7.5 | 7.5-20.7 | 20.7-27.3 | >27.3 |
| Hallux | Load in Kilo Pascal | — | — | 0.0-43.5 | 43.5-66.6 | >66.6 |
| | % of total foot load | — | — | 0-4 | 4-6.3 | >6.3 |
| Second toe | Load in Kilo Pascal | — | 0-0.3 | 0.3-17.1 | 17.1-25.5 | >25.5 |
| | % of total foot load | — | — | 0-0.8 | 0.8-1.2 | >1.2 |
| Lateral toe | Load in Kilo Pascal | — | 0-5.3 | 5.3-26.9 | 26.9-37.7 | >37.7 |
| | % of total foot load | — | 0-0.5 | 0.5-2.5 | 2.5-3.5 | >3.5 |

A patient specific device in which each region is made of a material having the provided durometer, is manufactured (step 104). Optionally, a durometer map is provided to a manufacturing machine such as a three-dimensional (3D) printer, which prints the device. In a non-limiting example, the 3D printer may print using a thermoplastic such as ethylene-vinyl acetate (EVA), plastazote, rubber like material, or nylon. In another non-limiting example, the 3D printer may print using viscoelastic material. Optionally, the 3D printing is of a composite of materials.

As used herein, the term “3D printer” or “manufacturing machine” refers to any such numerically controlled manufacturing machine, such as three-dimensional additive manufacturing machines configured for rapid prototyping, three-dimensional printing, two-dimensional printing, freeform fabrication, solid freeform fabrication, incremental sheet forming, and stereolithography. Manufacturing machines can also include a subtractive manufacturing machine, including machines adapted for milling, turning, and/or an additive manufacturing machine, and/or an injection molding machine. The manufacturing machines can further include an extrusion manufacturing machine, a melting manufacturing machine, a solidification manufacturing machine, an ejection manufacturing machine, a die casting manufacturing machine, a stamping process machine, an assembly robot assembling 3D objects from pieces or blocks.

The manufacturing instructions that control the manufacturing machines can be, e.g., G-codes or other instructions according to any computer language, including numerical control (CNC) programming language, but also high-level languages like python, java, PHP, etc. Such manufacturing instructions may define where to move to, how fast to move, and through what path to move the operative part of the manufacturing machine, such as the printing head, the extruder head, etc., as well as other manufacturing parameters.

According to some exemplary embodiments shown on Table 2, the device is printed based on durometer ranges computed according to the determined degree of load pressure for each plantar region as determined on Table 1. According to these examples, lower durometer ranges are selected for higher degrees of plantar pressures. Accordingly, in one exemplary embodiment, for a plantar region with very high pressure (relative to a normal individual), a low durometer material (e.g., 15 to 20 according to the 00 scale of ASTN D2240, Type A) may be selected; for a planter region with high pressure a medium durometer material (e.g., 20 to 25 according to that scale) may be selected; and for a plantar region with low pressure, a high durometer material (e.g., 40 to 45 according to that scale) may be selected (Table 2, Example 1). Further, a region corresponding to a plantar region which has been the subject of a previously lesion or of a current lesion, may be printed with the low durometer material.

TABLE 2
Durometer values per degree of load pressure

| Example | | | | | | |
|---|---|---|---|---|---|---|
| No. | Load pressure | Extra low | Low | Medium | High | Extra high |
| 1 | Durometer | 40-45 | 40-45 | 40-45 | 20-25 | 15-20 |
| 2 | Durometer | 40-55 | 40-55 | 40-55 | 25-30 | 15-25 |
| 3 | Durometer | 45-55 | 45-55 | 40-45 | 25-30 | 15-25 |
| 4 | Durometer | 50-55 | 45-50 | 40-55 | 25-30 | 20-25 |
| 5 | Durometer | 50-55 | 45-50 | 35-45 | 25-30 | 20-25 |
| 6 | Durometer | 45-55 | 45-55 | 45-55 | 30-40 | 15-25 |

Optionally, the device may vary in thickness. The device may have a maximum thickness, at one or more of its regions, of between 1 mm and 4 mm; alternatively, between 4 mm and 7 mm; alternatively, between 7 mm and 10 mm; alternatively, between 10 mm and 15 mm. Optionally, regions of the device corresponding to areas having an extra low and/or low pressure may have higher thickness than regions of the device corresponding to areas having medium pressure. As used herein, higher thickness is at least 20%, 30%, 40%, 50%, 60%, 70%, 80%, 90%, 100%, 110%, 120%, 130%, 140%, 150%, 160%, 170%, 180% or at least 200% increase in thickness. Optionally, regions of the device corresponding to areas having an extra high and/or high pressure may have lower thickness than regions of the device corresponding to areas having medium pressure. As used herein, lower thickness is at least 20%, 30%, 40%, 50%, 60%, 70%, 80%, or at least 90% decrease in thickness. Optionally, to prevent injuries, a borderline between regions of different thicknesses is smooth and continuous. In a non-limiting example, a thickness may vary gradually from a region of a first thickness to a region of a second thickness.

In another embodiment, pressure values (in kPa) deviate by 1-5% than pressures values presented in Table 1. Optionally, pressure values (in kPa) are all 1-5% higher than pressures values presented in Table 1. Alternatively, pressure values (in kPa) are all 1-5% lower than pressures values presented in Table 1.

In yet another embodiment, pressure values (in kPa) deviate by 5-10% than pressures values presented in table 1. Optionally, pressure values (in kPa) are all 5-10% higher than pressures values presented in table 1. Alternatively, pressure values (in kPa) are all 5-10% lower than pressures values presented in table 1.

There is further provided, in accordance with an embodiment, a computer program product for generating a patient specific device parameters for reducing plantar pressure, the computer program product comprising a non-transitory computer-readable storage medium having program code embodied therewith, the program code executable by at least one hardware processor to: provide a suitable material for each of a plurality of regions of the device, wherein the suitable material is provided according to a computed durometer range, wherein the durometer range is computed in accordance with a measured pressure of a corresponding plantar region.

The present invention or some aspects thereof may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

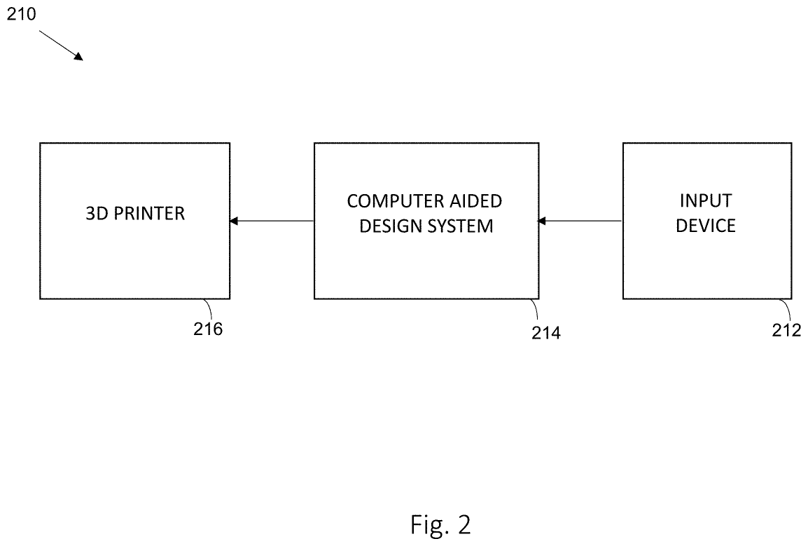

Reference is now made to FIG. 2 which shows a block diagram of a system 210 for manufacturing a patient specific device for reducing plantar pressure, in accordance with an embodiment. System 210 includes an input device 212 for entering patient's measurements into a database; a computer aided design system 214 for processing the measurements (e.g., pressure), calculating the patient specific parameters (e.g. a durometer range for a material for each plantar region) and preparing a computerized model of the patient-specific IAL; and a 3D printer 216 that prints a patient specific device according to the computerized model.

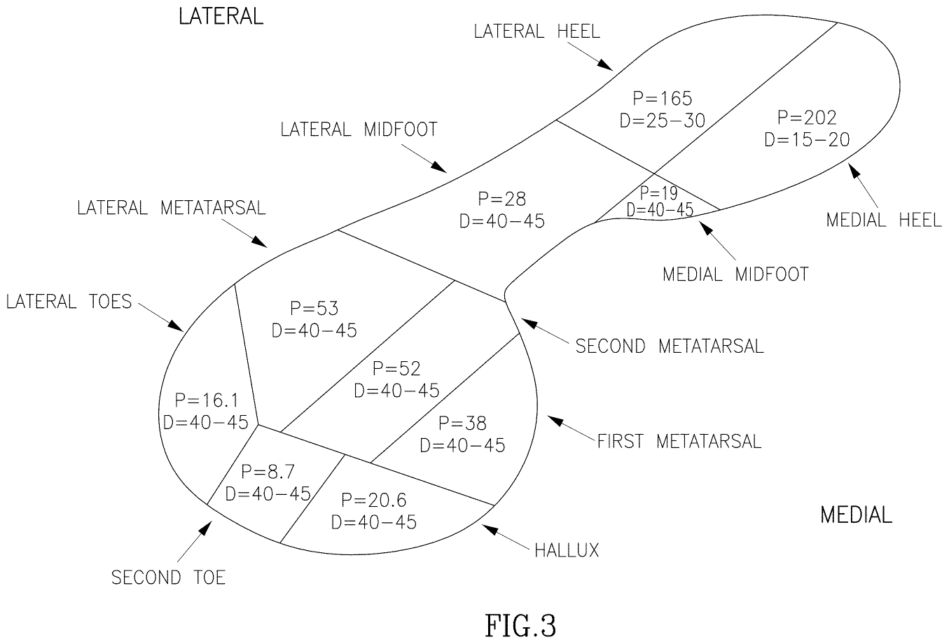

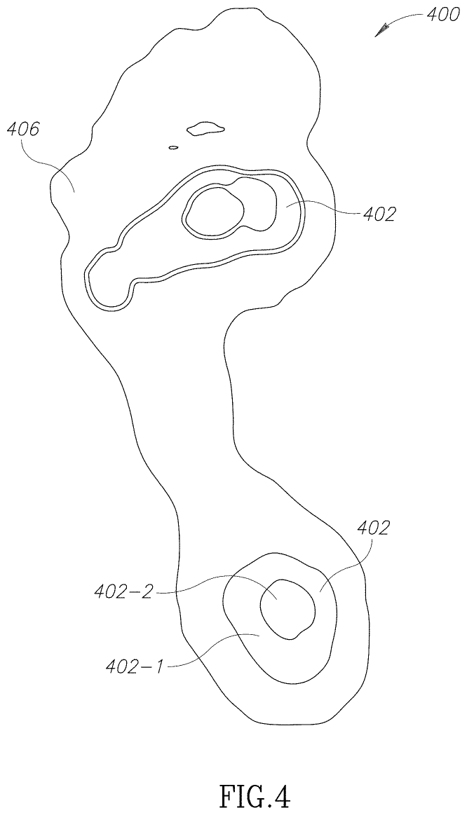

Reference is now made to FIG. 3 which shows a patient specific device 330 for reducing plantar pressure, manufactured according to the rules presented in Table 3, in accordance with an embodiment. Patient specific device 330 includes a plurality of regions 332, each of plurality of regions 332 is configured to contact a corresponding region of a plantar surface of a foot as indicated in FIG. 3 (medial heel, lateral heel, medial midfoot, lateral midfoot, first metatarsal, second metatarsal, lateral metatarsal, hallux, second toe, lateral toes). Each of plurality of regions 332 includes a material having a specific durometer range computed according to a patient specific pressure in a corresponding region of region of plantar surface of a foot as indicated. For each region of plurality of regions 332, the load pressure (P) in kPa of the corresponding region of the plantar surface of the foot is indicated. Additionally, for each region of plurality of regions 332, a selected durometer range (D) computed according to table 3, is indicated. In the exemplary embodiment, medial heel exhibit very high pressure (202 kPa), and lateral heel exhibits high pressure (165 kPa). Accordingly, a durometer of 15-20 is used in the region which is configured to contact the medial heal, and a durometer of 25-30 is used in the region which is configured to contact the lateral heal. In contrast, durometer of 40-45 are used in regions configured to contact the: medial midfoot, lateral midfoot, first metatarsal, second metatarsal, lateral metatarsal, hallux, second toe, lateral toes, which exhibit normal pressures.

TABLE 3
Durometer value per degree of load pressure

| | | Durometer | | | | |
|---|---|---|---|---|---|---|
| | | OO scale of ASTM D2240 | | | | |
| | | 40-55 | 40-55 | 40-55 | 25-30 | 15-20 |
| | | Load pressure | | | | |
| | | Extra | Low | | | |
| | | low | high | Medium | High | Extra |
| Medial | Load in Kilo Pascal | <76.1 | 76.1-107.5 | 107.5-170.3 | 170.3-201.7 | >201.7 |
| heel | % of total foot load | <15.3 | 15.3-23.9 | 23.9-41.1 | 41.1-49.7 | >49.7 |
| Lateral | Load in Kilo Pascal | <68 | 68-100.3 | 100.3-164.9 | 164.9-197.2 | >197.2 |
| heel | % of total foot load | 11.6 | 11.6-19.8 | 19.8-36.2 | 36.2-44.4 | >44.4 |
| Medial | Load in Kilo Pascal | — | 0-5.7 | 5.7-32.7 | 32.7-46.2 | >46.2 |
| midfoot | % of total foot load | — | 0-0.3 | 0.3-2.5 | 2.5-3.6 | >3.6 |
| Lateral | Load in Kilo Pascal | — | 0-13.2 | 13.2-42.4 | 42.4-57 | >57 |
| midfoot | % of total foot load | — | 0-1.5 | 1.5-11.3 | 11.3-16.2 | >16.2 |
| First | Load in Kilo Pascal | — | 0-14.4 | 14.4-62.4 | 62.4-86 | >86.4 |
| metatarsal | % of total foot load | — | 0-1.1 | 1.1-10.1 | 10.1-14.6 | >14.6 |
| Second | Load in Kilo Pascal | <8.6 | 8.6-30.2 | 30.2-73.4 | 73.4-95 | >95 |
| metatarsal | % of total foot load | <0.8 | 0.8-4.6 | 4.6-12.2 | 12.2-16 | >16 |
| Lateral | Load in Kilo Pascal | <9.8 | 9.8-31.6 | 31.6-75.2 | 75.2-97 | >97 |
| metatarsal | % of total foot load | <0.9 | 0.9-7.5 | 7.5-20.7 | 20.7-27.3 | >27.3 |
| Hallux | Load in Kilo Pascal | — | — | 0.0-43.5 | 43.5-66.6 | >66.6 |
| | % of total foot load | — | — | 0-4 | 4-6.3 | >6.3 |
| Second toe | Load in Kilo Pascal | — | 0-0.3 | 0.3-17.1 | 17.1-25.5 | >25.5 |
| | % of total foot load | — | — | 0-0.8 | 0.8-1.2 | >1.2 |
| Lateral toe | Load in Kilo Pascal | — | 0-5.3 | 5.3-26.9 | 26.9-37.7 | >37.7 |
| | % of total foot load | — | 0-0.5 | 0.5-2.5 | 2.5-3.5 | >3.5 |

Optionally, each of the different regions of the device may be independently formed of meshes, foams, or gels for obtaining specific durometers. Optionally, other properties such as strength, stiffness, resilience, toughness, and density may be further considered to achieve the desired performance of the patient specific device of reducing plantar pressure in a patient.

Optionally, the patient specific device may be formed of a plurality of layers stacked one on top of another. In a non-limiting example, the layers may be independently formed of meshes, foams, or gels. Optionally, at least one layer may be formed of a net or a mesh of fibers with designed spaces (“mesh layer”). Optionally, the patient specific device may be formed of a multi-layer net or mesh of fibers with designed spaces (“mesh layer”). Non-limiting examples of suitable materials for the fibers include: plastic, nitinol, stainless steel, nylon, HDPE, polyethylene, cobalt based alloys, PEEK and the like. Optionally, thicknesses of the wires may vary between different portions/areas of the mesh layer according to the patient specific parameters. Optionally, sizes of designed spaces may vary between different portions/areas of the mesh layer according to the patient specific parameters. Optionally, the spaces of the mesh may be impregnated with materials such as in a form of a gel or a foam. In a non-limiting example, the spaces are impregnated with a gel (e.g., a silicone gel) to give support to the gel and increase its stiffness and fatigue endurance limit. Optionally, the patient specific device may further include ventilation holes which allow air to flow between a top portion and a bottom portion of the patient specific device.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The device or IAL as described herein is utilized for balancing gait of a user suffering from a lower limb pathology and/or a defective gait parameter. The device or IAL as described herein is utilized for exerting the least valgus, varus, dorsal or plantar torque about the ankle in a user. The device or IAL as described herein is utilized for providing the least or minimal lower limbs muscle activity.

The device or IAL as described herein can be utilized for tuning a lower limbs muscle activity. The device or IAL as described herein can be utilized for toning a lower limb muscle. The device or IAL as described herein can be utilized for toning the amount of tension or resistance to movement in a muscle involved in gait.

The device or IAL as described herein can be utilized for extending the mobility of a user. The device or IAL as described herein can be utilized for maximal ankle, knee, and hip joint mobility. The device or IAL as described herein can be utilized for providing a differential reduction of a muscle activity, larger passive ankle excursion, improved gait ability, or any combination thereof.

The device or IAL as described herein can be utilized for increasing step length, stance symmetry, or a combination thereof. The device or IAL as described herein can be utilized for increasing the length of the force point of action in lower limb muscles such as but not limited to: soleus, tibialis posterior, and both gastrocnemius muscles.

The device or IAL as described herein can be utilized for correction of early heel-rise in both right and left. The device or IAL as described herein can be utilized for treating lower limb pain such as but not limited to bi-lateral patello-femoral pain syndrome.

The device or IAL as described herein can be useful, for example, for plantar sores caused by diseases, e.g. diabetes, or orthopedic conditions, e.g., lateral meniscus tear or damage, lateral compartment knee osteoarthritis, valgus knee (genu valgus), patello-femoral pain syndrome, patello-femoral problem (malalignment), Medial collateral Ligament tear, bone bruise or avascular necrosis of the lateral tibial plateau or lateral femoral condyle hip labrum damage or tear, hip pain, ankle instability (pronoation), achilles tendonitis, tibilias insufficiency and metatarsalgia.

According to an aspect of some embodiments of the invention there is provided an insole adaptive layer. In some embodiments, the insole adaptive layer comprises a surface comprising a plurality of contour lines, wherein the contour lines are defining specific zones. In some embodiments, the insole adaptive layer surface within each zone has predetermined mechanical properties specific to the zone.

In some embodiments, the boundary lines of each of the zones corresponds to contour lines obtained from pedobarographic data of a subject. In some embodiments, the mechanical properties include at least one or combination of Shore hardness, young's modulus, shear modulus, yield stress, compression, thickness, elasticity, and ductility. In some embodiments, the insole adaptive layer is used within a shoe to engage the sole of a subject's foot.

According to an aspect of some embodiments of the invention there is provided an insole adaptive layer in which the values of the mechanical properties of the surface of at least one zone of the insole adaptive layer are inverse in relation to the pressure values of at least one region in the pedobarographic data.

According to an aspect of some embodiments of the invention there is provided an insole adaptive layer wherein at least one surface zone of the insole adaptive layer is configured to succumb to pressure applied by a corresponding plantar pressure zone of a subject. In some embodiments, the zone of the insole adaptive layer which is configured to succumb to pressure is configured to engage at least a portion of the plantar zone.

In some embodiments, the zone of the insole adaptive layer which is configured to succumb to pressure is larger than the portion of the plantar zone with which it engages. In some embodiments, the zone of the insole adaptive layer which is configured to succumb to pressure remains engaged to the portion of the plantar zone during shifting of the plantar zone in respect to the insole adaptive layer.

According to an aspect of some embodiments of the invention there is provided a system for manufacturing an insole. In some embodiments, the system comprises at least one sensor used to obtain pedobarographic data of a subject. In some embodiments, the system comprises a processor in communication with the sensor. In some embodiments, the system comprises a manufacturing device operative to manufacture the insole. In some embodiments, the processor comprises a computer program product configured to record zones defined by the pedobarographic data.

In some embodiments, the processor comprises a computer program product configured to allot Shore hardness values to the zones in accordance with the pedobarographic data. In some embodiments, the processor comprises a computer program product configured to generate a map associating the allotted Shore hardness values with the recorded zones. In some embodiments, the manufacturing device is configured to produce a surface of an insole comprising zones of different Shore hardness in accordance with the map.

According to an aspect of some embodiments of the invention there is provided a method for manufacturing an insole. In some embodiments, the method comprises generating pedobarographic data of a sole of a subject applied to a surface. In some embodiments, the method comprises recording zones defined by said pedobarographic data. In some embodiments, the method comprises allotting Shore hardness values to said zones in accordance with said pedobarographic data. In some embodiments, the method comprises generating a map associating said allotted Shore hardness values with said recorded zones. In some embodiments, the method comprises printing a surface of an insole comprising zones of different Shore hardness in accordance with said map.

According to an aspect of some embodiments of the invention there is provided a method for converting a prefabricated shoe insole into a personalized custom insole by applying to the shoe insole an insole adaptive layer (IAL) that is based on the patient's peak dynamic pressure map. In some embodiments, the IAL is fixed to the upper portion of the insole and then covered with a top cover which when added relieves pressure from high peak dynamic plantar pressure areas. In some embodiments, the IAL is added to a specific area of interest only on the insole where there is clinical interest to relieve plantar peak dynamic pressures.

Reference is now made to FIG. 4, which shows a plan view simplified illustration of an insole adaptive layer in accordance with some embodiments of the invention. In some embodiments, the Insole Adaptive Layer (IAL) 400 is configured to be placed onto a shoe insole. In some embodiments, the IAL is used to accommodate a sole of a subject while using the shoe. In some embodiments, the IAL 400 is a sheet. In some embodiments, the IAL 400 thickness varies. In some embodiments, the IAL 400 thickness is 0.02-6 mm. In some embodiments, the IAL thickness is 5-20 mm. In some embodiments, the IAL thickness is 10-50 mm. As described in greater details elsewhere herein, in some embodiments, the IAL 400 is dimensioned to be placed onto a shoe insole inside a shoe. In some embodiments, the IAL 400 is dimensioned to be placed onto a portion of a shoe insole inside a shoe.

In some embodiments, the IAL 400 comprises a plurality of zones 402. In some embodiments, the zones 402 are marked on at least one surface of the IAL 400. In some embodiments, the zones 402 are visually indistinguishable to a user. In some embodiments, a boundary line 404 circumscribes a group of zones 402. In some embodiments, the zone boundary lines 404 are visually indistinguishable. In some embodiments, the zone boundary lines 404 are marked onto at least one surface of the IAL 400. In some embodiments, some of the zones 402 are surrounded by other zones 402. For example, in the embodiment depicted by FIG. 4, zone 402-1 surrounds zone 402-2.

In some embodiments, each of the zones 402 has specific mechanical properties. In some embodiments, one of the mechanical properties is Shore hardness. In some embodiments, each of the zones 402 has specific Shore hardness. In some embodiments, the Shore hardness of the zones 402 ranges between 00-10 to D-70. In some embodiments, one of the mechanical properties is young's modulus. In some embodiments, each of the zones 402 has specific young's modulus. In some embodiments, the young's modulus of each of the zones 402 ranges between 0.1-20 GPa. In some embodiments, each of the zones 402 has specific yield stress. In some embodiments, the yield stress of each of the zones 402 ranges between 30-150 MPa. In some embodiments, each of the zones 402 has specific shear modulus. In some embodiments, the shear modulus of each of the zones 402 ranges between 0.0001-3 GPa. In some embodiments, the shear modulus of each of the zones 402 ranges between 0.1-30 GPa.

In some embodiments, the IAL 400 is composed of one or more materials. In some embodiments, the IAL 400 is composed of at least two materials, wherein each material has distinct mechanical properties. In some embodiments, each of the zones 402 comprises different materials. In some embodiments, each of the zones 402 comprises a combination of materials. In some embodiments, the combination of materials is in the form of a mixture. In some embodiments, the combination of materials is in the form of alternating layers.

In some embodiments, the zones 402 differ from one another in at least one or a combination of density, thickness, morphology, and color. In some embodiments, the thickness of at least one zone 402 ranges between 0.01-2 mm. in some embodiments, the thickness of at least one zone 402 ranges between 0.6-4 mm. in some embodiments, the thickness of at least one zone 402 ranges between 1-5.5 mm.

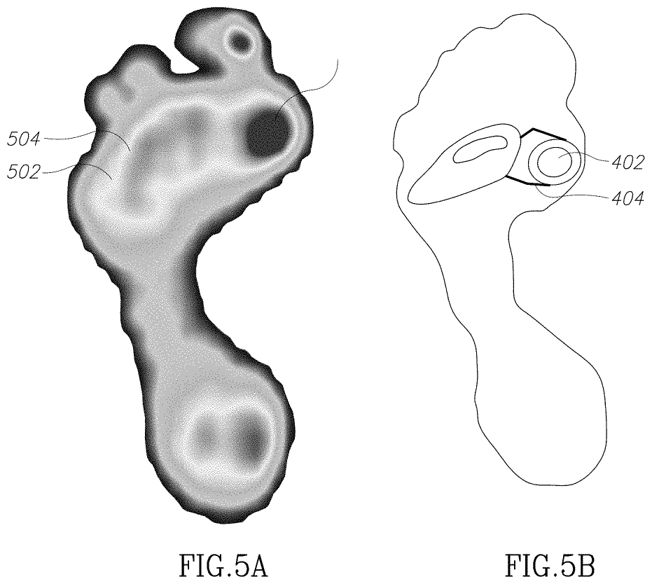

Reference is made to FIGS. 5A and 5B, collectively referred to as FIG. 5, which show a plan view simplified illustration of an insole adaptive layer corresponding with data of a pedobarographic measurement in accordance with some embodiments of the invention. As described in greater detail elsewhere herein, in some embodiments, the mechanical properties of the zones 402 in the IAL 400 are determined in accordance with pedobarographic data obtained from a subject. Typically, systems that map plantar pressure are composed of sensor elements. The actual sensors can be either capacitive, resistive, piezoelectric or piezoresistive, to name a few examples. Some systems report the actual numerical pressure at each sensor point, either in kilo Pascal or kilo Pascal/cm², or expressed within specific pressure ranges. Commercially available systems include, for example: The F-Scan System by Tekscan, Inc, of Boston, Mass.; and the Stepscan® Pedway by ViTRAK Systems Inc., Canada. In some embodiments, the pedobarographic data is obtained from a 3D scanner, e.g., Rscan® by RSscan International NV, Belgium. In some embodiments, the pedobarographic data is dynamic pedobarographic data obtained from a subject while in motion (e.g., walking). In some embodiments, such as depicted by FIG. 5A, the obtained pedobarographic data 500 is in the form of a plantar pressure distribution map. In some embodiments, the pedobarographic data 500 comprises contour lines 502. In some embodiments, the contour intervals between the couture lines 502 represent a difference in pressure between the different contour lines 502. In some embodiments, the contour intervals range between 0-600 kPa.

In some embodiments, the zone boundary lines 404 of the IAL 400 correspond to the contour lines 502 indicated by the pedobarographic data 500. In some embodiments, the shapes of the zone boundary lines 404 in the IAL 400 are identical to the shapes of the contour lines 502 of the pedobarographic data 500. In some embodiments, the shapes of the zone boundary lines 404 in the IAL 400 are identical to the shapes of the corresponding contour lines 502 of the pedobarographic data 500. In some embodiments, each contour line 502 of the pedobarographic data 500 has a corresponding boundary line 404 of one or more zones 402 in the IAL 400. In some embodiments, each contour line 502 of the pedobarographic data 500 encloses an area 504. In some embodiments, each of the zones 402 in the IAL 400 correspond to an area 504 of the pedobarographic data 500. In some embodiments, each zone 402 corresponding to a specific area 504 is identical in shape and size to the corresponding area 504 in the pedobarographic data 500. In some embodiments, each zone 402 corresponding to a specific area 504 is larger than its corresponding area 504 in the pedobarographic data 500. In some embodiments, each zone 402 corresponding to a specific area 504 is at least 2-15% larger than its corresponding area 504 in the pedobarographic data 500. In some embodiments, each zone 402 corresponding to a specific area 504 is at least 15-50% larger than its corresponding area 504 in the pedobarographic data 500. In some embodiments, the perimeter of each zone boundary 404 corresponding to a contour line 502 is larger than the perimeter of its corresponding area 504 in the pedobarographic data 500. In some embodiments, the perimeter of each zone boundary 404 corresponding to a contour line 502 is similar to the perimeter of its corresponding area 504 in the pedobarographic data 500.

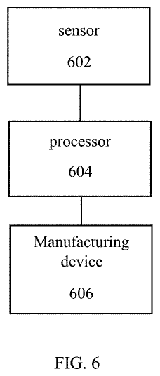

Reference is made to FIG. 6, which shows a system for producing an Insole Adaptive Layer (IAL). In some embodiments, there is provided a system 600 for producing an IAL. In some embodiments, the system 600 comprises at least one sensor 602. As described in greater detail elsewhere herein, in some embodiments, at least one sensor 602 is in communication with a processor 604. In some embodiments, the processor 604 is in communication with a manufacturing device 606.

In some embodiments, at least one sensor 602 is used to obtain pedobarographic data from a subject. In some embodiments, the sensor 602 is a pressure sensor. In some embodiments, the sensor 602 is a temperature sensor. the sensor 602 is an IR sensor. In some embodiments, the sensor 602 is used to obtain dynamic pedobarographic data. In some embodiments, the sensor 602 is used to obtain static pedobarographic data. In some embodiments, the processor 604 comprises a computer program product configured to record zones 504 defined by the pedobarographic data.

In some embodiments, the processor 604 comprises a computer program product configured to allot values corresponding to mechanical properties of production materials with the pedobarographic data. In some embodiments, the processor 604 comprises a computer program product configured to generate a map associating the designated values of mechanical properties of production materials with the recorded zones. In some embodiments, the production materials are used to produce the IAL 400. In some embodiments, the processor 604 communicates the production of the IAL 400 data to a manufacturing device 606. In some embodiments, the manufacturing device 606 is a 3D printer.

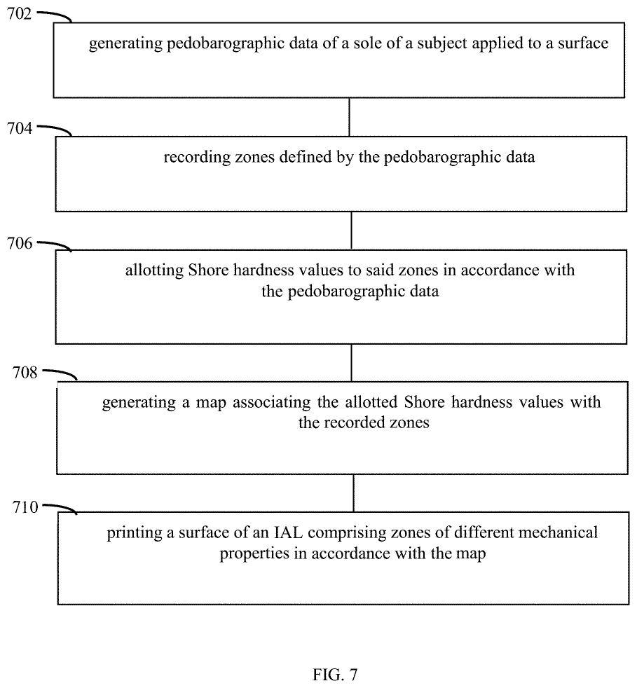

Reference is made to FIG. 7, which is a flow chart depicting a method for manufacturing an insole adaptive layer. In some embodiments, the method comprises generating at 702 pedobarographic data of a sole of a subject applied to a surface. In some embodiments, the method comprises at 704 recording zones defined by said pedobarographic data. In some embodiments, the method comprises designating at 706 Shore hardness values to the zones in accordance with the pedobarographic data. In some embodiments, the method comprises at 708 generating a map associating the designated Shore hardness values with the recorded zones. In some embodiments, at 710 the method comprises printing a surface of an IAL comprising zones of different mechanical properties in accordance with the map.

In some embodiments, the system 600 is used to manufacture patient specific IAL. In some embodiments, the system 600 is used to manufacture patient specific IAL by obtaining pedobarographic data from a specific patient. In some embodiments, the system 600 is used to manufacture a universal IAL. In some embodiments, the system 600 is used to manufacture a universal IAL based on averages of obtained pedobarographic data.

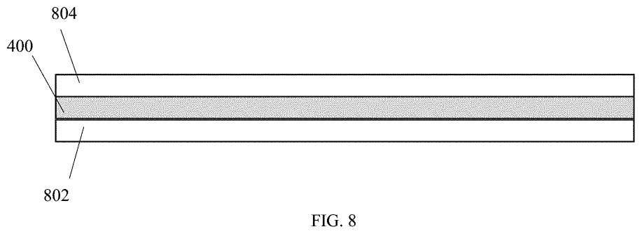

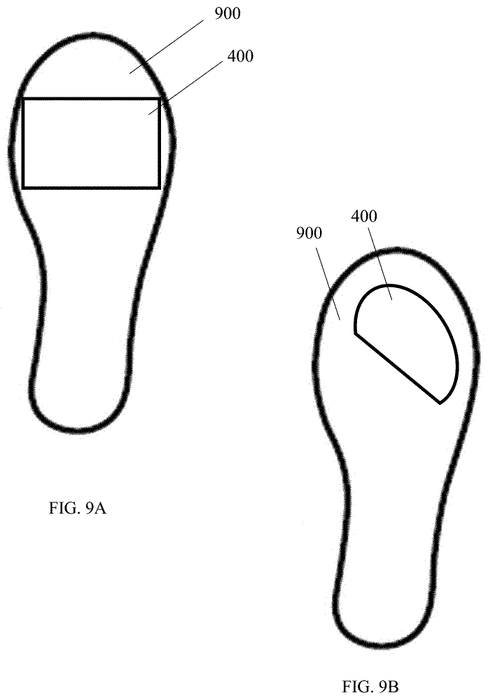

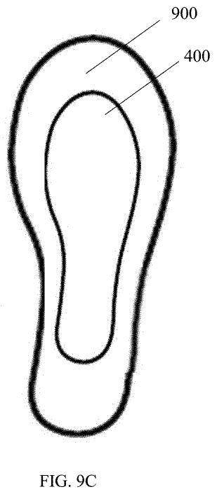

Reference is made to FIG. 8, which is a side view simplified illustration of a shoe insole comprising an insole adaptive layer in accordance with some embodiments of the invention. In some embodiments, the IAL 400 is placed onto a base 802. In some embodiments, the base 802 is a shoe insole. In some embodiments, a cover layer 804 is placed onto the IAL 400. In some embodiments, the cover layer 804 is flexible. In some embodiments, the cover layer 804 is made of a pliable material (e.g., Neoprene®, rubber, cellulose, cloth). In some embodiments, the IAL 400 is adhered to at least one of the base 802 or cover 804. In some embodiments, the IAL 400 is placed between the base 802 and cover 804. In some embodiments, the IAL 400 is placed on an insole of a shoe. In some embodiments, the IAL 400, base 802 and/or the cover 804 are placed in a shoe. In some embodiments, the IAL 400 replaces a shoe insole. In some embodiments, and as shown in the exemplary embodiment depicted in FIGS. 9A-9C, which are plan view simplified illustrations of an insole adaptive layer in accordance with some embodiments of the invention, the IAL 400 covers at least a portion of an insole 900.

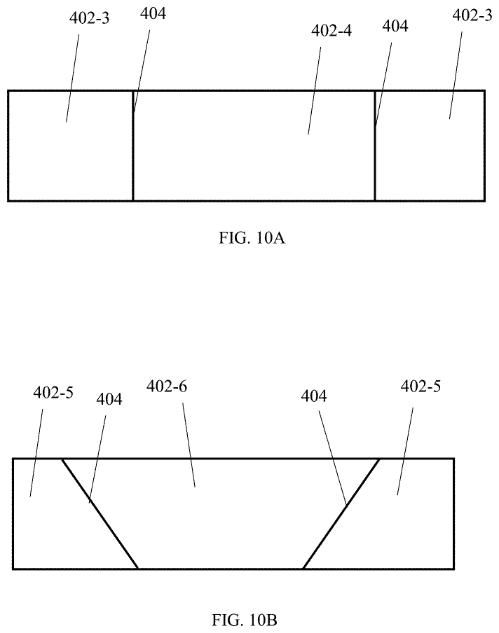

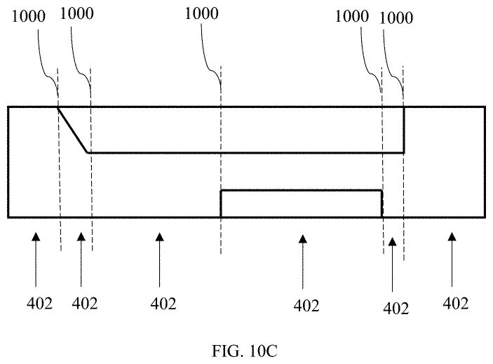

Reference is made to FIG. 10A-C, which are cross section view simplified illustrations of an insole adaptive layer in accordance with some embodiments of the invention. In some embodiments, such as depicted in FIG. 10A, the boundary lines 404 of each zone 402 are perpendicular at least one surface 406 of the IAL. In some embodiments, each of the zones comprises a different material. In some embodiments, each of the zones comprises a different combination of materials. In some embodiments, each of the zones comprises a different configuration of the one material. In some embodiments, as depicted by FIG. 10B, the boundary lines 404 of the zones 402 are non-perpendicular with the surface 406 of the IAL. In some embodiments, such as depicted by FIG. 10C, the boundary lines 404 of each zone 402 are virtual lines 1000 separating at least two regions of the IAL which comprise different mechanical properties. in some embodiments, such as depicted by FIG. 10C, the IAL comprises segments 1002 of materials.

In some embodiments, the IAL 400 is composed of a plurality of materials. In some embodiments, the materials are layered. In some embodiments, the different materials are mixed. In some embodiments, the IAL 400 is composed of at least one material that has a Shore hardness in the range of 00-10 to A-60. In some embodiments, the IAL 400 is composed of at least one material that has a Shore hardness in the range of A-50-10 to D-100.

In some embodiments, the IAL is composed of a combination of at least one two materials, where one material has a Shore hardness in the range of 00-10 to A-60 and the other has a Shore hardness in the range of A-50-10 to D-100. In some embodiments, the IAL is composed of at least one combination of materials having a cumulative Shore hardness in the range A-27-A-95. In some embodiments, the IAL is composed of at least one combination of materials having a cumulative Shore hardness in the range 00-10-A-100. In some embodiments, the different materials are layered. In some embodiments, the different materials are layered in varying thicknesses. In some embodiments, the IAL comprises a plurality of zones 402 configured to succumb to pressure applied by a corresponding plantar pressure zone on a subject's sole placed on said insole. In some embodiments, the Shore hardness of the surface of a zone 402 corresponding to a high-pressure region of the pedobarographic data is lower than the Shore hardness of the surface of a zone 402 corresponding to a low pressure region of the pedobarographic data. In some embodiments, the Shore hardness of a zone 402 is inversely related to the pressure measurement of the corresponding region of the pedobarographic data.

In some embodiments, the IAL comprises various areas having different mechanical properties for engaging at least one of plantar region of a patient. In one example, an IAL can be manufactured for subjects who suffers plantar ulcers (e.g., diabetics), wherein the IAL of the patient includes zones 402 made to engage with the plantar areas containing the ulcers. In this example, the zones engaging with the plantar areas containing the ulcers have a lower Shore hardness and higher elasticity than the surrounding zones 402 of the IAL which engage with the healthy portions of the subject's foot. In this example, the IAL relieves some of the pressure applied by an insole to ulcers within weight-bearing plantar areas.

In another example, an IAL can be manufactured for subjects who suffer from plantar corns, wherein the IAL of the patient includes zones 402 made to engage with the plantar areas containing the corns. In this example, the plantar areas of the subject can be obtained using a scanner, e.g., CT-scan. In this example, the IAL includes zones 402 made to engage with the plantar areas containing the corns. In this example, the zones engaging with the plantar areas containing the corns have a higher degree of deformation than the surrounding zones 402 of the IAL which engage with the healthy portions of the subject's foot. In this example, the IAL relieves some of the pressure applied by an insole to corns within weight-bearing plantar areas.

In some embodiments, the IAL 400 comprises zones 402 which do not directly correspond with the obtained pedobarographic data 500. In some embodiments, the IAL 400 comprises zones 402 configured to shift the center of pressure of the subject's foot.

A potential advantage of the boundary lines 404 of the zones 402 in the IAL 400 being larger than their corresponding contour lines 502 of the pedobarographic data 500 is in that at least a portion of a zone 402 remains engaged with at least a portion of a subject's foot, while the foot shifts (e.g., slides) over the IAL.

Under normal circumstances, a shoe is designed to allow some shifting of a foot over the shoe insole during walking. The IAL is designed to be used in a regular shoe which is worn on a subject's foot and is therefore designed and manufactured such that zones 402 remain engaged with a desired specific portion of the subject's foot during the shifting of the food in relation to the shoe insole.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An insole adaptive layer (IAL), comprising:
a surface comprising a plurality of contour lines, said contour lines defining zones; wherein
said surface within each zone has predetermined mechanical properties specific to said zone; and
said contour lines of each of said surface zones correspond to contour lines obtained from pedobarographic data of a subject; wherein
a perimeter of at least one of said zones is larger than a corresponding plantar zone of a subject such that said plantar zone placed on said insole adaptive later remains engaged with said insole adaptive layer zone during shifting of said plantar zone in respect to said insole adaptive layer.

2. The insole adaptive layer according to claim 1, wherein said mechanical properties include at least one or a combination of Shore hardness, young's modulus, shear modulus, yield stress, compression, thickness, elasticity, and ductility.

3. The insole adaptive layer according to claim 1, wherein at least one of said surface zones is configured to succumb to pressure applied by a corresponding plantar pressure zone of a subject.

4. The insole adaptive layer according to claim 3, wherein a degree of deformation of said surface within said surface zone corresponds to a value of pressure applied by said plantar pressure zone as expressed by said pedobarographic data of said subject.

5. The insole adaptive layer according to claim 1, wherein values of said mechanical properties of at least one zone of the surface are inversely related to pressure values of at least one corresponding region defined by said pedobarographic data.

6. The insole adaptive layer according to claim 1, wherein at least one of said zones is least deformable and supports over 50% of the plantar pressure applied by a sole of a subject.

7. The insole adaptive layer according to claim 1, wherein the mechanical properties of each zone vary throughout a depth of said zone.

8-10. (canceled)

11. The insole adaptive layer according to claim 1, wherein the surface is composed of one or more materials.

12. The insole adaptive layer according to claim 11, wherein at least one material has a Shore hardness in the range of 00-10 to A-60.

13-14. (canceled)

15. The insole adaptive layer according to claim 11, wherein said surface comprises one or more materials arranged in layers of varying thicknesses, having a cumulative Shore hardness of one or more of A-27, A-40, A-50, A-60, D-0, D-10, A-70, D-20, A-85, A-9.

16. A method for manufacturing an insole adaptive layer, comprising:
generating pedobarographic data of a sole of a subject;
recording zones defined by said pedobarographic data;
designating Shore hardness values to said zones in accordance with said pedobarographic data;
generating a model, in which each zone is at least 2-15% larger than its corresponding recorded zone defined by said pedobarographic data;
associating said designated Shore hardness values with said recorded zones; and
printing a surface of an insole adaptive layer comprising zones of different Shore hardness in accordance with said model.

17. The method according to claim 16 wherein printing at least one layer using a plurality of materials.

18. A system for manufacturing an insole adaptive layer comprising:
at least one sensor used to obtain pedobarographic data of a subject;
a processor in communication with said sensor;
a manufacturing device operative to manufacture said insole adaptive layer; wherein said processor comprises a computer program product configured to
record zones defined by said pedobarographic data;
allot Shore hardness values to said zones in accordance with said pedobarographic data; and
generate a model in which each zone is at least 2-15% larger than its corresponding recorded zone defined by said pedobarographic data;
associate said designated Shore hardness values with said recorded zones; and
wherein said manufacturing device is configured to produce a surface of an insole adaptive layer comprising zones of different Shore hardness in accordance with said model.

19. (canceled)

20. The system according to claim 18 wherein said manufacturing device is configured to produce zones in which mechanical properties of said surface within said zone are distinct.

21. (canceled)

22. The method according to claim 1, wherein said plurality of plantar regions is selected from the group consisting of: medial heel, lateral heel, medial midfoot, lateral midfoot, first metatarsal, second metatarsal, lateral metatarsal, hallux, second toe, lateral toe.

23. (canceled)

24. The insole adaptive layer of claim 1, wherein a medial heel degree of load pressure differs from differs from the midfoot degree of load pressure.

25-31. (canceled)
